(19) United States
(12) Patent Application Publication
(10) Pub. No.: US 2026/0022318 A1
(43) Pub. Date: Jan. 22, 2026

(54) CELL CULTURE CONTAINER

(71) Applicant: Sharp Display Technology Corporation, Kameyama City (JP)

(72) Inventors: Chihiro TACHINO, Kameyama City (JP); Satoshi IHIDA, Kameyama City (JP); Tomoko TERANISHI, Kameyama City (JP)

(21) Appl. No.: 19/274,713

(22) Filed: Jul. 21, 2025

(30) Foreign Application Priority Data
Jul. 22, 2024 (JP) 2024-116796

(51) Int. Cl. C12M 1/12; C12M 1/00; C12M 1/42

(52) U.S. Cl. CPC C12M 25/04; C12M 23/20; C12M 35/02

(57) ABSTRACT

A cell culture container includes an upper surface and a lower surface located opposite to the upper surface, a recessed portion formed in the upper surface, and a channel that is in communication with the recessed portion. The recessed portion has a lateral surface that includes a slope portion inclined with respect to the upper surface and a bottom surface that is generally parallel to the upper surface or the lower surface. The channel includes a first portion extending in a direction parallel to the upper surface or the lower surface and located on the lower surface side of the bottom surface, and a second portion connecting the first portion with an opening formed in the bottom surface.

### BACKGROUND

### 1. Technical Field

The present invention relates to cell culture containers.

### 2. Description of the Related Art

In recent years, for drug discovery research and other purposes, there has been a growing demand to culture various types of cells in vitro. Among others, nerve cells have a characteristic morphology, which consists of a cell body, an axon (a projection growing from the cell body) and dendrites. The longest one of projections growing from the cell body of a nerve cell is the axon. To improve biological consistency with neural tissues in vivo, culture methods have been developed in which the axon growing from the cell body is allowed to extend away from the cell body such that the cell body and the axon are separated from each other.

WO 2022/261775 and Japanese Patent No. 6430680 disclose cell culture devices for use in culturing nerve cells. The cell culture devices of WO 2022/261775 and Japanese Patent No. 6430680 are configured such that the cell body of a nerve cell and the axon growing from the cell body are received in separate parts. The cell culture device of WO 2022/261775 has a part that receives a liquid culture medium and a channel connected to the part that receives the liquid culture medium, the channel having a part that receives the cell body and a part that receives the axon. The part that receives the axon is located at an opposite side of the part that receives the cell body from the part that receives the liquid culture medium, and is configured such that the axon extends away from the part that receives the liquid culture medium. The cell culture device of Japanese Patent No. 6430680 includes a culture module that has two recessed portions (chambers) and a channel connecting these recessed portions. The culture module of the cell culture device of Japanese Patent No. 6430680 is configured such that the cell body of a nerve cell is placed in one of the two recessed portions and that the axon growing from the cell body is formed in the channel.

There is a need to culture nerve cells more efficiently.

The present invention intends to provide a cell culture container in which nerve cells can be efficiently cultured.

### SUMMARY

According to embodiments of the present invention, the solutions described in the following items are provided.

### Item 1

A cell culture container including:
an upper surface and a lower surface located opposite to the upper surface;
a recessed portion formed in the upper surface, the recessed portion having a bottom surface that is generally parallel to the upper surface or the lower surface and a lateral surface that includes a slope portion inclined with respect to the bottom surface; and
a channel that is in communication with the recessed portion,
wherein the channel includes
   a first portion extending in a direction parallel to the bottom surface and located on the lower surface side of the bottom surface, and
   a second portion connecting the first portion with an opening formed in the bottom surface.

### Item 2

The cell culture container of Item 1, wherein the second portion is extending in a direction intersecting the bottom surface.

### Item 3

The cell culture container of Item 1 or 2, wherein the second portion is located so as to overlap the bottom surface as viewed from above.

### Item 4

The cell culture container of any one of Items 1 to 3, wherein the lateral surface has one or more protrusions protruding toward an inside of the recessed portion.

### Item 5

The cell culture container of Item 4, wherein the protrusion extends from the lateral surface in a direction parallel to the bottom surface or toward the upper surface side.

### Item 6

The cell culture container of Item 4 or 5, wherein
the one or more protrusions are a plurality of protrusions, and
the plurality of protrusions are located with equal intervals at an intersection of the lateral surface and a predetermined plane that is parallel to the bottom surface.

### Item 7

The cell culture container of Item 6, wherein the plurality of protrusions include a pair of protrusions that are symmetrical about a center of the recessed portion as viewed from above.

### Item 8

The cell culture container of any one of Items 4 to 7, wherein the protrusion is integrally formed with the lateral surface.

### Item 9

The cell culture container of any one of Items 4 to 7, further comprising a supporting portion that is located in contact with the lateral surface of the recessed portion and that is conformable to the lateral surface,
wherein the protrusion is supported by the supporting portion.

### Item 10

The cell culture container of any one of Items 1 to 9, wherein
the lateral surface includes a first portion and a second portion located on the upper surface side of the first portion of the lateral surface, and
the second portion of the lateral surface has lower cell adhesiveness than the first portion of the lateral surface.

### Item 11

The cell culture container of Item 10, wherein
the second portion of the lateral surface has a minute uneven structure at its surface, and
the first portion of the lateral surface does not have a minute uneven structure at its surface.

### Item 12

The cell culture container of Item 10 or 11, wherein
the first portion of the lateral surface has a cell adhesion layer, which has cell adhesiveness, at its surface, and
the second portion of the lateral surface does not have the cell adhesion layer at its surface.

### Item 13

The cell culture container of Item 10, wherein a part including the second portion of the lateral surface is made of a material that has lower cell adhesiveness than a part including the first portion of the lateral surface.

### Item 14

The cell culture container of any one of Items 1 to 13, further comprising one or more electrodes located in the first portion of the channel.

### Item 15

The cell culture container of any one of Items 1 to 14, further comprising
a base having an upper surface, and
a well member supported by the upper surface of the base, the well member including the recessed portion and the channel,
wherein the upper surface of the base forms a bottom of the first portion of the channel.

According to an embodiment of the present invention, a cell culture container is provided in which nerve cells can be efficiently cultured.

### BRIEF DESCRIPTION OF THE DRAWINGS

FIG. 1 schematically shows a top view and a cross-sectional view of a cell culture container 50A according to an embodiment of the present invention.

FIG. 2A is a schematic cross-sectional view for illustrating a nerve cell culture method with the use of the cell culture container 50A.

FIG. 2B is a schematic cross-sectional view for illustrating a nerve cell culture method with the use of the cell culture container 50A.

FIG. 2C is a schematic cross-sectional view for illustrating a nerve cell culture method with the use of the cell culture container 50A.

FIG. 2D is a schematic cross-sectional view for illustrating a nerve cell culture method with the use of the cell culture container 50A.

FIG. 3A is a schematic cross-sectional view for illustrating a nerve cell culture method with the use of a cell culture container 950A of a comparative example.

FIG. 3B is a schematic cross-sectional view for illustrating a nerve cell culture method with the use of the cell culture container 950A of the comparative example.

FIG. 4 is a schematic cross-sectional view for illustrating a nerve cell culture method with the use of a cell culture container 950B of another comparative example.

FIG. 5A is a schematic cross-sectional view of the cell culture container 50A.

FIG. 5B is a schematic cross-sectional view of a cell culture container 50A1 according to a variation example of the embodiment of the present invention.

FIG. 6A is a schematic cross-sectional view of a cell culture container 50B according to another embodiment of the present invention.

FIG. 6B is a schematic top view of a recessed portion 32 of the cell culture container 50B.

FIG. 6C is a schematic top view of the recessed portion 32, which shows another example of the protrusions 44 of the cell culture container 50B.

FIG. 6D is a schematic top view of the recessed portion 32, which shows another example of the protrusions 44 of the cell culture container 50B.

FIG. 6E is a schematic cross-sectional view of the cell culture container 50B, which shows another example of the protrusions 44 of the cell culture container 50B.

FIG. 7 is a schematic cross-sectional view showing an example of a nerve cell culture process with the use of the cell culture container 50A.

FIG. 8A is a schematic cross-sectional view of the cell culture container 50B1 according to a variation example of another embodiment of the present invention.

FIG. 8B is a schematic top view of the recessed portion 32 of the cell culture container 50B1.

FIG. 9 is a schematic cross-sectional view of a cell culture container 50C according to still another embodiment of the present invention.

FIG. 10 is a schematic cross-sectional view of a cell culture container 50C1 according to a variation example of still another embodiment of the present invention.

FIG. 11 is a schematic cross-sectional view of a cell culture container 50D according to still another embodiment of the present invention.

### DETAILED DESCRIPTION

Hereinafter, cell culture containers according to embodiments of the present invention are described with reference to the drawings. Note that the present invention is not limited to the embodiments exemplified in the following sections. In the drawings mentioned below, components that have substantially the same function are indicated by common reference symbols, and the descriptions thereof may be omitted.

### Embodiment 1

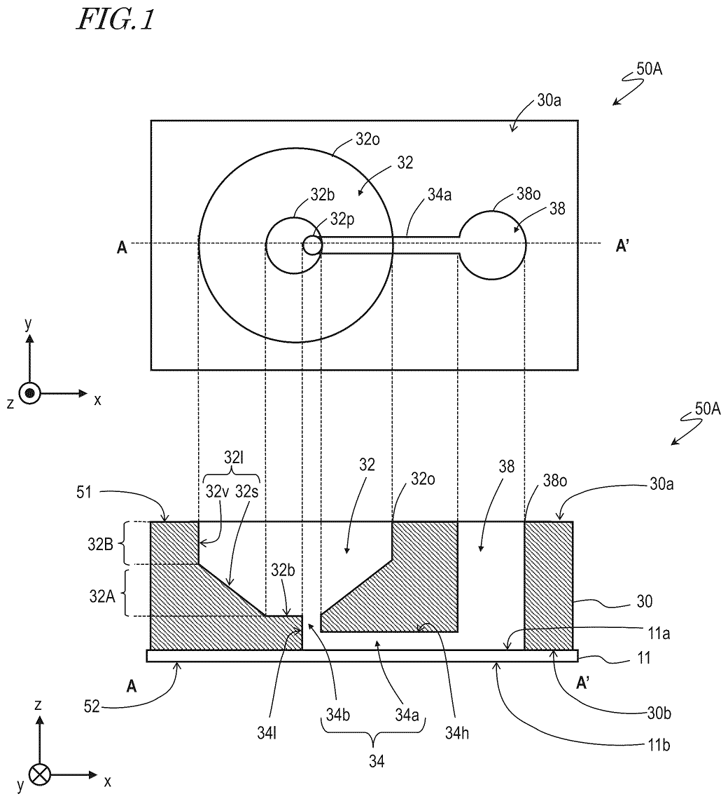

FIG. 1 schematically shows a top view and a cross-sectional view of a cell culture container 50A according to an embodiment of the present invention. The upper part of FIG. 1 schematically shows the top view of the cell culture container 50A, and the lower part of FIG. 1 schematically shows a cross-sectional view taken along line A-A′ of the upper part of FIG. 1.

The cell culture container 50A has an upper surface 51 and a lower surface 52 located opposite to the upper surface 51, a recessed portion 32 formed in the upper surface 51, and a channel 34 that is in communication with the recessed portion 32. In this example, the cell culture container 50A includes a base 11 and a well member 30 supported by the base 11.

The base 11 has an upper surface 11a and a lower surface 11b, which are two main surfaces located opposite to each other. The upper surface 11a and the lower surface 11b of the base 11 are, for example, generally parallel to each other. The upper surface 11a and the lower surface 11b of the base 11 are, for example, generally parallel to the xy plane. The well member 30 is supported by the upper surface 11a of the base 11. In this example, the lower surface 52 of the cell culture container 50A includes the lower surface 11b of the base 11.

The base 11 is made of, for example, glass or a resin material. The base 11 may be required that at least the upper surface 11a is made of a low cytotoxic material. The base 11 may be made of a material of high visible light transmittance. Examples of the material of the base 11 which has low cytotoxicity and high visible light transmittance include glass, polycarbonate, PDMS (polydimethylsiloxane), COP (cyclo olefin polymer), and PET (polyethylene terephthalate). When the base 11 has high visible light transmittance, it is possible to observe cells in the cell culture container 50A (e.g., cells during the culture process) using an inverted microscope. The visible light transmittance of the base 11 may be, for example, 80% or higher, or may be 90% or higher.

The well member 30 has a lower surface 30b on the base 11 side and an upper surface 30a located opposite to the lower surface 30b. The lower surface 30b of the well member 30 is, for example, generally parallel to the upper surface 11a of the base 11. The lower surface 30b of the well member 30 is, for example, generally parallel to the xy plane. The upper surface 30a of the well member 30 may be generally parallel to the lower surface 30b of the well member 30 or may be intersecting the lower surface 30b of the well member 30. In this example, the upper surface 51 of the cell culture container 50A includes the upper surface 30a of the well member 30.

The well member 30 can be made of a low cytotoxic material (e.g., resin material, resist, or the like). The well member 30 may also be made of a material of high visible light transmittance. The well member 30 can be made of the same material as the resin material of the base 11 described above. For example, the well member 30, which has the configuration and shape described below, can be formed using known injection molding or photolithography techniques.

The base 11 and the well member 30 may be adhered together via an adhesive material. Examples of the adhesive material include UV curable resins, epoxy resins, and double-sided tapes. Preferably, the adhesive material is a highly biocompatible material. Also, preferably, the adhesive material is a highly water-resistant material. More preferably, the adhesive material has both high biocompatibility and high water-resistance. Alternatively, the base 11 and the well member 30 may be bound together without using an adhesive agent, for example, by performing a surface treatment, specifically plasma treatment, on the upper surface 11a of the base 11 and the lower surface 30b of the well member 30. Examples of the materials of the base 11 and the well member 30 which can undergo the plasma treatment include glass, PDMS, and COP.

The well member 30 includes a recessed portion 32 formed in the upper surface 30a (i.e., the upper surface 51 of the cell culture container 50A) and a channel 34 that is in communication with the recessed portion 32. That is, the opening 32o of the recessed portion 32 is formed in the upper surface 30a.

The recessed portion 32 includes a bottom surface 32b that is generally parallel to the upper surface 51 of the cell culture container 50A (the upper surface 30a of the well member 30) or the lower surface 52 (the lower surface 11b of the base 11), and a lateral surface 32l that includes a slope portion 32s inclined with respect to the bottom surface 32b. The bottom surface 32b is a flat surface that is generally parallel to the xy plane, for example. The bottom surface 32b may be, for example, generally parallel to the lower surface 30b of the well member 30 or the upper surface 11a of the base 11. In the thickness direction of the well member 30 (z direction in the drawings), the bottom surface 32b is located between the lower surface 30b and the upper surface 30a. That is, in the thickness direction of the well member 30, the distance between the bottom surface 32b and the upper surface 30a is smaller than the distance between the lower surface 30b and the upper surface 30a. In this example, the shape of the bottom surface 32b is generally circular. Note that, however, the shape of the bottom surface 32b is not limited to this example but may be polygonal, for example. The bottom surface 32b has an opening 32p that is in communication with the channel 34. The opening 32p may be provided at an edge of the bottom surface 32b as in the shown example, or may be provided at the central part of the bottom surface 32b.

The slope portion 32s of the lateral surface 32l is configured such that the cross-sectional area of the recessed portion 32 at a plane perpendicular to the normal direction of the upper surface 30a (the cross-sectional area in the xy plane in the drawing) is greater at a position more distant from the bottom surface 32b. In this example, the shape of the slope portion 32s in cross-sectional view is linear. Note that, however, the shape of the slope portion 32s in cross-sectional view is not limited to linear shapes but may have a shape which includes steps (e.g., stair-like shape) or may have a shape which includes curves.

In this example, the lateral surface 32l further includes a vertical portion 32v that is perpendicular to the upper surface 30a in addition to the slope portion 32s. In the shown example, the recessed portion 32 includes a first portion 32A that includes the slope portion 32s at the lateral surface and a second portion 32B that includes the vertical portion 32v at the lateral surface. The first portion 32A includes the bottom surface 32b of the recessed portion 32, and the second portion 32B includes the opening 32o of the recessed portion 32. In this example, the first portion 32A has the shape of a generally circular truncated cone, and the second portion 32B has the shape of a generally circular cylinder. The shapes of the first portion 32A and the second portion 32B of the recessed portion 32 are not limited to the shown example. The second portion 32B may be omitted.

The channel 34 includes a first portion 34a and a second portion 34b. The first portion 34a is extending in a direction parallel to the bottom surface 32b (in this example, in a direction parallel to the xy plane in the drawing) and located on the base 11 side of the bottom surface 32b. The first portion 34a is defined by the upper surface 11a of the base 11 and the inner wall surface 34h that is opposite to the upper surface 11a. That is, the upper surface 11a of the base 11 forms the bottom of the first portion 34a of the channel 34. The second portion 34b connects the first portion 34a and the opening 32p provided in the bottom surface 32b. The second portion 34b is extending in a direction intersecting (in this example, perpendicular to) the bottom surface 32b. The second portion 34b is located so as to at least partially overlap the bottom surface 32b as viewed from above. The second portion 34b includes an inner wall surface 34l that is perpendicular to the bottom surface 32b. The inner wall surface 34l may be inclined with respect to the bottom surface 32b. The bottom surface 32b of the recessed portion 32 is connected with the inner wall surface 34l of the second portion 34b of the channel 34, so that a step is formed between the bottom surface 32b and the inner wall surface 34l. Herein, the “step” includes flexures or discontinuity points between the bottom surface 32b and the inner wall surface 34l.

In this example, the well member 30 further includes a through hole 38 that is in communication with the channel 34. The through hole 38 has an opening in each of the upper surface 30a and the lower surface 30b of the well member 30. The opening 38o in the upper surface 30a of the through hole 38 does not overlap the opening 32o of the recessed portion 32. In this example, the through hole 38 has the shape of a generally circular cylinder. Note that, however, the shape of the through hole 38 is not limited to this example but may be, for example, polygonal.

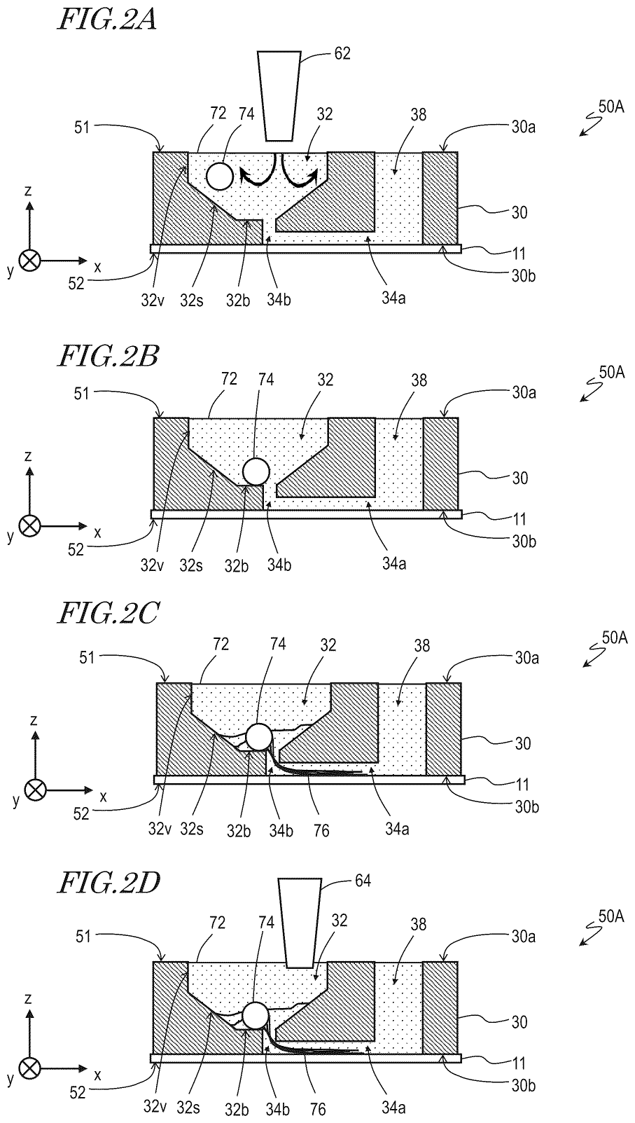

FIG. 2A, FIG. 2B, FIG. 2C and FIG. 2D are schematic cross-sectional views for illustrating a nerve cell culture method with the use of the cell culture container 50A. The reasons why the use of the cell culture container 50A enables efficient culture of nerve cells will be described with reference to FIG. 2A, FIG. 2B, FIG. 2C and FIG. 2D.

As described in the following, nerve cell aggregates (i.e. clumps of the cell bodies of nerve cells) are seeded into the cell culture container 50A and cultured, so that axons growing from the cell bodies can self-organize into a bundle. Culturing with the use of the cell culture container 50A results in nerve cell aggregates with bundled axons. Note that, in culturing nerve cells with the use of the cell culture container 50A, part of the surfaces of the cell culture container 50A to which the cells can adhere (e.g., surfaces which are in the recessed portion 32, in the channel 34, and in the through hole 38) may be treated with a coating solution containing preference ingredients for which the cells exhibit a preference, such as extracellular matrix (ECM).

As shown in FIG. 2A, a nerve cell aggregate 74 is transferred (seeded) into the cell culture container 50A from, for example, another container using a pipette 62. In this process, the recessed portion 32 of the cell culture container 50A is filled with a liquid culture medium (or culture solution) 72. From the pipette 62, the liquid culture medium containing the aggregate 74 is dropped onto the liquid culture medium 72 in the recessed portion 32. Dropping of the liquid culture medium containing the aggregate 74 from the pipette 62 results in formation of flow in the liquid culture medium 72 in the recessed portion 32 as shown by the bold arrows in FIG. 2A. The aggregate 74 floats along this flow in the liquid culture medium 72 in the recessed portion 32. Since nerve cells are adhesive cells, they need to attach to a scaffold for proliferation and growth. That is, nerve cells are inactive while they are floating in the liquid culture medium 72 so that axons cannot grow.

As shown in FIG. 2B, as the flow in the liquid culture medium 72 in the recessed portion 32 slows down, the aggregate 74 sinks down in the liquid culture medium 72 in the recessed portion 32 to land on and adhere to the bottom surface 32b of the recessed portion 32. In this process, since the lateral surface 32l of the recessed portion 32 includes the slope portion 32s, the aggregate 74 can move along the slope portion 32s to the bottom surface 32b and be guided to the bottom surface 32b. That is, it is possible to increase the probability that the aggregate 74 adheres to the bottom surface 32b. Note that, preferably, the bottom surface 32b of the recessed portion 32 is treated with a coating solution containing preference ingredients for which the cells exhibit a preference such as described above.

As shown in FIG. 2C, the aggregate 74 is kept adhered onto the bottom surface 32b while the culture process is carried out. As described above, there is a step formed between the bottom surface 32b and the inner wall surface 34l of the second portion 34b of the channel 34. Proliferation and growth of adhesive cells are promoted on surfaces having an uneven structure (e.g. including raised portions, recessed portions, projections, steps, etc.) rather than on flat surfaces. Therefore, when the aggregate 74 is kept adhered onto the bottom surface 32b while the culture is carried out, the culture of the aggregate 74 can be efficiently carried out.

Further, when the cell culture container 50A is used, the direction of growth of axons 76 can be easily controlled during the culture process. At least some of the axons 76 growing from the aggregate 74 extend into the channel 34 via the opening 32p provided in the bottom surface 32b. The axons 76 extend away from the aggregate 74 along the extension direction of the first portion 34a of the channel 34 and form a bundle in the first portion 34a of the channel 34. Since the aggregate 74 and the axons 76 can be separately cultured in this way, the biological consistency with neural tissues in the body can be improved. Note that some of the axons 76 growing from the aggregate 74 can be formed in the recessed portion 32 as shown in the drawings.

Further, when the cell culture container 50A is used, the liquid culture medium 72 easily reaches the area under the aggregate 74 as compared with a case where the aggregate 74 is cultured on the upper surface 11a of the base 11, for example, as in a comparative example which will be described later. Therefore, the nutrients in the liquid culture medium 72 easily reach the aggregate 74. Thus, the cell culture efficiency can improve.

As shown in FIG. 2D, during the culture process, the step of replacing the liquid culture medium 72 in the recessed portion 32 (also referred to as “medium replacing step”) can be further performed. Specifically, the step of supplying (adding) a liquid culture medium into the recessed portion 32 and, furthermore, the step of sucking out part of the liquid culture medium 72 from the recessed portion 32 before supplying (adding) a liquid culture medium are performed when necessary during the culture process. The cells consume necessary nutrients from the liquid culture medium 72 and excrete waste products into the liquid culture medium 72. Therefore, preferably, the above-described medium replacing step is performed when the proportion of nutrients in the liquid culture medium 72 decreases during the culture process and/or the waste products in the liquid culture medium 72 increase during the culture process.

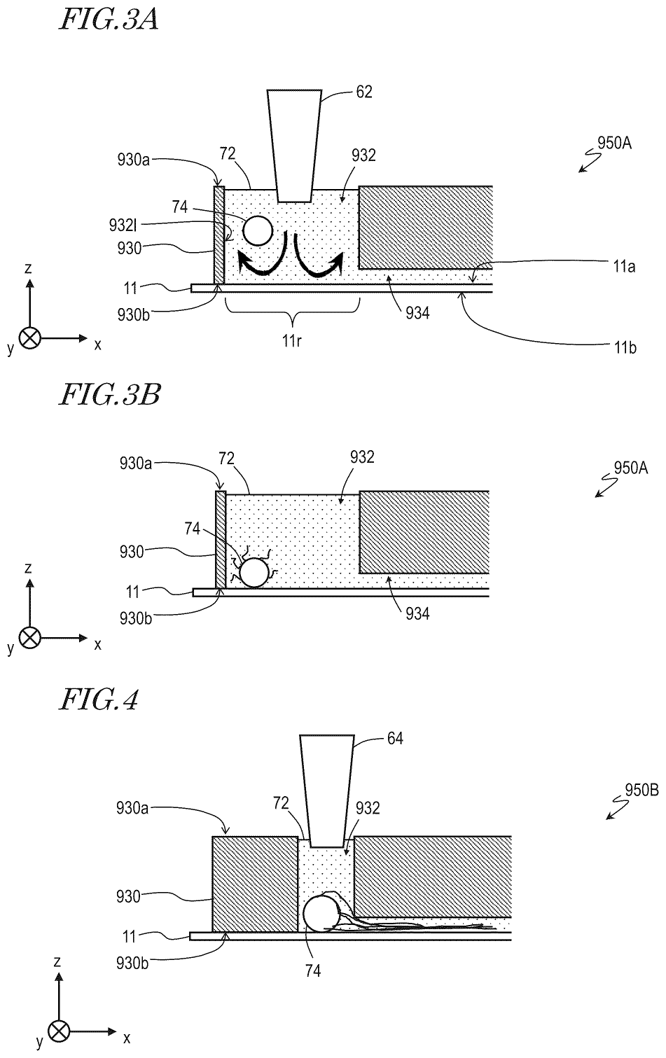

FIG. 3A and FIG. 3B are schematic cross-sectional views for illustrating a cell culture container 950A of a comparative example and a nerve cell culture method with the use of the cell culture container 950A. The differences from the cell culture container 50A of the present embodiment and the cell culture method with the use of the cell culture container 50A are mainly described.

As shown in FIG. 3A and FIG. 3B, the cell culture container 950A of the comparative example is different in the configuration and shape of the well member 930 from the cell culture container 50A that includes the well member 30. The well member 930 has a through hole 932, which has openings in the upper surface 930a and the lower surface 930b, and a channel 934 that is in communication with the through hole 932. The lateral surface 932l of the through hole 932 is perpendicular to the upper surface 930a and does not include a portion inclined with respect to the upper surface 930a. That is, the through hole 932 has the shape of a generally circular cylinder or a prismatic shape. The through hole 932 exposes a first region 11r that is a part of the upper surface 11a of the base 11.

The nerve cell culture method with the use of the cell culture container 950A of the comparative example is described.

As shown in FIG. 3A, a nerve cell aggregate 74 is transferred (seeded) into the cell culture container 950A of the comparative example from, for example, another container using a pipette 62. From the pipette 62, the liquid culture medium containing the aggregate 74 is dropped onto the liquid culture medium 72 in the through hole 932. Dropping of the liquid culture medium containing the aggregate 74 from the pipette 62 results in formation of flow in the liquid culture medium 72 in the through hole 932 as shown by the bold arrows in FIG. 3A. The aggregate 74 floats along this flow in the liquid culture medium 72 in the through hole 932.

As shown in FIG. 3B, as the flow in the liquid culture medium 72 slows down, the aggregate 74 sinks down in the liquid culture medium 72 in the through hole 932 to land on and adhere to the first region 11r of the upper surface 11a of the base 11 which is exposed through the through hole 932. In this process, the position in the first region 11r to which the aggregate 74 adheres is randomly determined. From the viewpoint of allowing the axons growing from the aggregate 74 to extend along a direction in which the channel 934 extends, it is preferred that the aggregate 74 adheres to part of the first region 11r in the vicinity of the channel 934. However, as shown in FIG. 3B, when the aggregate 74 adheres at a position distant from the channel 934, it is sometimes difficult to form the axons growing from the aggregate 74 in the channel 934. In such a case, the direction of growth of the axons cannot be controlled so that the axons sometimes cannot successfully form a bundle.

In contrast, according to the cell culture container 50A of the present embodiment and the cell culture method with the use of the cell culture container 50A, as previously described, it is possible to increase the probability that the aggregate 74 adheres to the bottom surface 32b and, therefore, the problems in the cell culture container 950A of the comparative example and the cell culture method with the use of the cell culture container 950A can be solved.

FIG. 4 is a schematic cross-sectional view for illustrating a cell culture container 950B of another comparative example and a nerve cell culture method with the use of the cell culture container 950B. The differences from the cell culture container 950A and the cell culture method with the use of the cell culture container 950A are mainly described.

As shown in FIG. 4, in the cell culture container 950B of another comparative example, the area as viewed from above of the first region 11r of the upper surface 11a of the base 11 which is exposed through the through hole 932 is small as compared with the cell culture container 950A. When the cell culture container 950B is used in place of the cell culture container 950A, the position at which the aggregate 74 adheres can be easily controlled so as to be located in part of the first region 11r in the vicinity of the channel 934. However, as shown in FIG. 4, when the cell culture container 950B is used, at the medium replacing step during the culture process, the distance between the aggregate 74 and a pipette 64 for culture medium replacement is short and, accordingly, the probability that the pipette 64 unintendedly sucks in the aggregate 74 while sucking in the liquid culture medium 72, or the probability that the aggregate 74 is affected by the flow caused when adding the liquid culture medium from the pipette 64 into the through hole 932, increases. If such occurs, the cell culture efficiency will decrease. Alternatively, if the above-described probability is decreased, the efficiency of the culture medium replacement can decrease.

In contrast, according to the cell culture container 50A and the cell culture method with the use of the cell culture container 50A, the problems in the cell culture container 950B of the comparative example and the cell culture method with the use of the cell culture container 950B can be solved. In the cell culture container 50A, the lateral surface 32l of the recessed portion 32 includes the slope portion 32s and, therefore, the area of the opening 32o of the recessed portion 32 is greater than the area of the bottom surface 32b of the recessed portion 32. Thus, in the medium replacing step of the cell culture method with the use of the cell culture container 50A, which is illustrated in FIG. 2D, the culture medium replacement can be carried out while the distance between the pipette 64 for the culture medium replacement and the aggregate 74 is maintained. When the cell culture container 50A is used, the effects (damage) on the aggregate 74 in the medium replacing step can be reduced without decreasing the efficiency of the culture medium replacement.

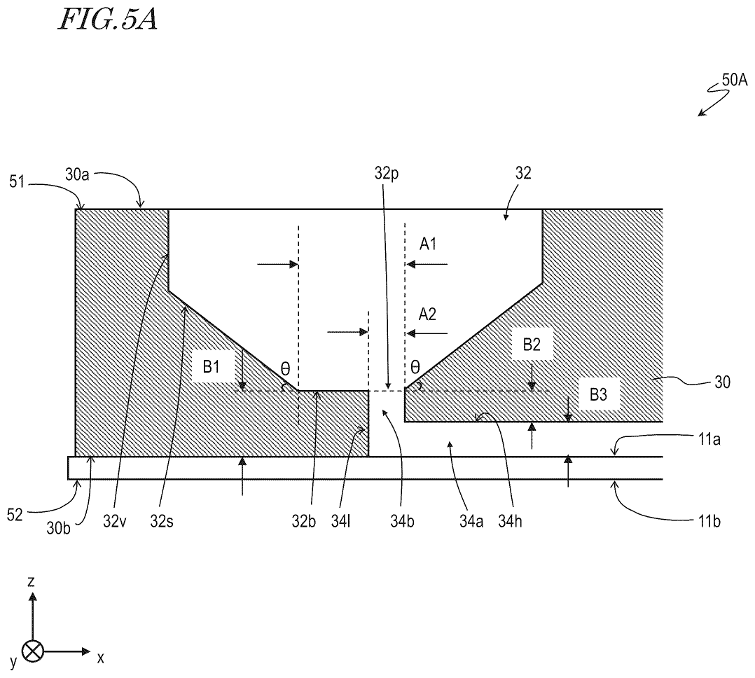

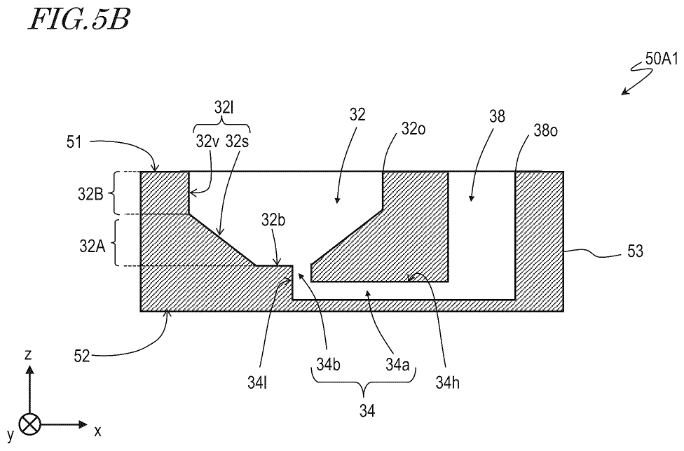

The shape and preferred size of the cell culture container 50A are described with reference to FIG. 5A. FIG. 5A is a schematic cross-sectional view of the cell culture container 50A.

The diameter A1 of the bottom surface 32b of the recessed portion 32 is, for example, about 500 μm. The diameter A1 of the bottom surface 32b of the recessed portion 32 can be, for example, equal to or greater than 300 μm and equal to or smaller than 500 μm. The diameter A1 of the bottom surface 32b of the recessed portion 32 can be, for example, equal to or greater than 1.5 times and equal to or smaller than 2.5 times the diameter of the cell body of nerve cells (or nerve cell aggregate) seeded into the cell culture container 50A. Herein, the diameter of the nerve cell aggregate in the cell culture container 50A is, for example, about 200 μm, although the diameter of the nerve cell aggregate is, for example, equal to or greater than 5 μm and equal to or smaller than 1 mm. When the shape of the bottom surface 32b as viewed from above is not circular, the area equivalent diameter of the bottom surface 32b as viewed from above can be set within the range of the diameter A1 mentioned above.

The diameter A2 of the opening 32p in the bottom surface 32b is, for example, equal to or smaller than 200 μm. For example, the diameter A2 may be equal to or greater than 0.1 times and equal to or smaller than 0.5 times the diameter A1 of the bottom surface 32b of the recessed portion 32. When the shape of the opening 32p as viewed from above is not circular, the area equivalent diameter of the opening 32p as viewed from above can be set within the range of the diameter A2 mentioned above.

The distance B1 in the thickness direction of the well member 30 between the bottom surface 32b of the recessed portion 32 and the upper surface 11a of the base 11 is, for example, equal to or greater than 10 μm and equal to or smaller than 100 μm. It can also be said that the distance B1 is the distance in the thickness direction of the well member 30 between the bottom surface 32b of the recessed portion 32 and the lower surface 30b of the well member 30. Since the distance B1 is equal to or greater than 10 μm, a step is formed between the bottom surface 32b and the inner wall surface 34l of the second portion 34b of the channel 34 and, therefore, the cell culture can be efficiently carried out.

The distance B2 in the thickness direction of the well member 30 between the bottom surface 32b of the recessed portion 32 and the inner wall surface 34h of the first portion 34a of the channel 34 is, for example, equal to or greater than 0 μm and equal to or smaller than 100 μm. The height B3 (the distance in the thickness direction of the well member 30) of the first portion 34a of the channel 34 is, for example, the value resulting from subtraction of the distance B2 from the distance B1 (B3=B1−B2). The height B3 of the first portion 34a is, for example, equal to or greater than 10 μm and equal to or smaller than 100 μm. The distance B2 and the height B3 can be appropriately set according to the diameter of the axon bundle formed in the first portion 34a of the channel 34. The diameter of the axon bundle is, for example, about 50 μm.

The slope angle θ of the slope portion 32s of the lateral surface 32l of the recessed portion 32 is, for example, equal to or greater than 20° and equal to or smaller than 80°. The slope angle of the slope portion 32s can be, in cross-sectional view (in the xz plane of the drawings), the angle between the line segment extending between the lower end and the upper end of the slope portion 32s and a straight line extending parallel to the upper surface 30a of the well member 30. Even when the shape of the slope portion 32s in cross-sectional view is not linear, the slope angle of the slope portion 32s can be determined in the same way.

### Variation Example

A cell culture container 50A1 according to a variation example of the present embodiment is described with reference to FIG. 5B. FIG. 5B is a schematic cross-sectional view of the cell culture container 50A1.

The cell culture container 50A1 is different from the cell culture container 50A, which includes the base 11 and the well member 30, in that the cell culture container 50A1 includes a well member 53. The well member 53 has such a shape and a configuration that the base 11 and the well member 30 of the cell culture container 50A are integrally formed.

The cell culture container 50A1 also achieves the same effects as those achieved by the cell culture container 50A.

### Embodiment 2

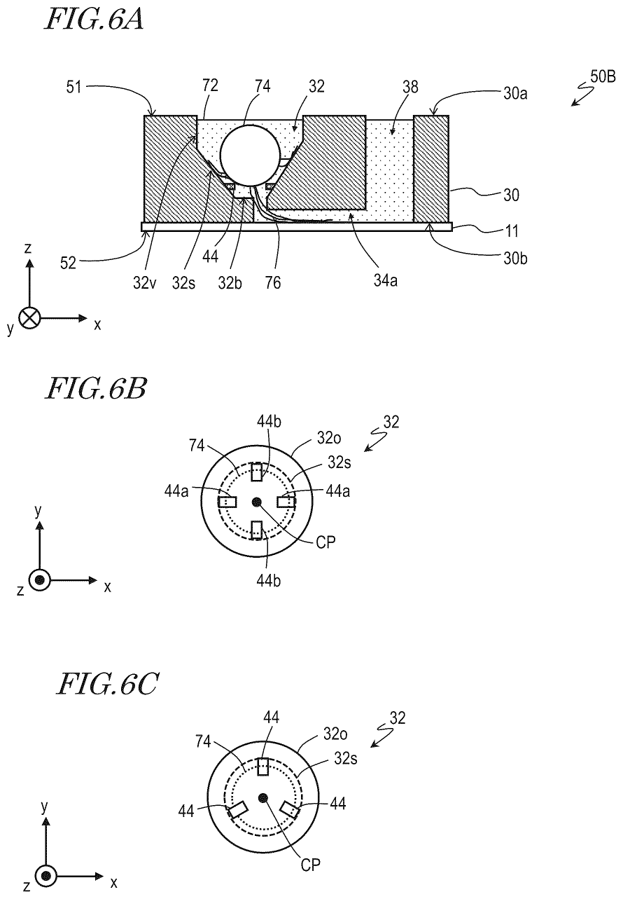

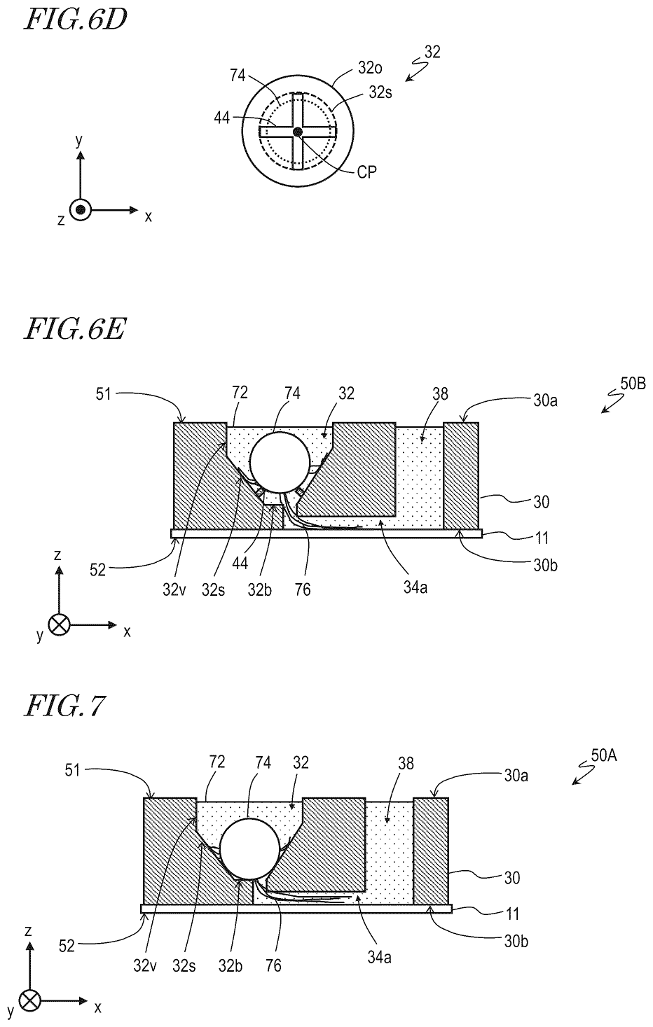

A cell culture container 50B of the present embodiment is described with reference to FIG. 6A and FIG. 6B. FIG. 6A is a schematic cross-sectional view of the cell culture container 50B, and FIG. 6B is a schematic top view of a recessed portion 32 of the cell culture container 50B. Hereinafter, the differences from the previously-described embodiment are mainly described.

The cell culture container 50B is different from the cell culture container 50A in that the lateral surface 32l of the recessed portion 32 has protrusions 44 protruding toward the inside of the recessed portion 32. In the shown example, the lateral surface 32l of the recessed portion 32 has a plurality of protrusions 44, although the lateral surface 32l may have at least one protrusion 44. Each of the protrusions 44 extends from the lateral surface 32l of the recessed portion 32 in a direction parallel to the bottom surface 32b (in this example, in a direction parallel to the xy plane in the drawing). In the shown example, the plurality of protrusions 44 are located with equal intervals at the intersection of the lateral surface 32l and a predetermined plane that is parallel to the bottom surface 32b (in this example, a plane parallel to the xy plane). In FIG. 6B, the intersection of the slope portion 32s of the lateral surface 32l and the plane parallel to the bottom surface 32b is indicated by a broken circle. As shown in FIG. 6B, the plurality of protrusions 44 (in this example, four protrusions 44) are located on the lateral surface 32l of the recessed portion 32 along the circumference of a circle that is the intersection of the lateral surface 32l and a plane parallel to the bottom surface 32b with equal intervals (in this example, at every 90° central angles). The plurality of protrusions 44 include a pair of protrusions 44a that are symmetrical about the center CP of the recessed portion 32 as viewed from above. In the shown example, the plurality of protrusions 44 further include a pair of protrusions 44b that are symmetrical about the center CP of the recessed portion 32 as viewed from above. One or more protrusions 44 are integrally formed with the lateral surface 32l of the recessed portion 32 using, for example, known injection molding techniques.

The cell culture container 50B also enables efficient culture of nerve cells as well as the cell culture container 50A of Embodiment 1.

Further, since the cell culture container 50B has the protrusions 44, the diameter of the nerve cell aggregate 74 that can be cultured in the cell culture container 50B has a wide variety of options (wide range) as compared with the cell culture container 50A that has no protrusions. As shown in FIG. 7, in the cell culture container 50A, if the diameter of the aggregate 74 is excessively large, the opening 32p in the bottom surface 32b of the recessed portion 32 is at least partially filled with (blocked by) the aggregate 74 so that, in some cases, transfer of the liquid culture medium 72 between the recessed portion 32 and the channel 34 via the opening 32p cannot be smooth. In such a case, for example, even if the medium replacing step is performed, the liquid culture medium 72 cannot be sufficiently replenished with nutrients, and waste products cannot be sufficiently removed from the liquid culture medium 72, so that the culture efficiency can decrease. Alternatively, in the case where the liquid culture medium 72 is sufficiently replenished with nutrients and waste products are sufficiently removed from the liquid culture medium 72, the workability of the culture medium replacement can decrease. In contrast, in the cell culture container 50B, the protrusions 44 can retain the aggregate 74 at a position closer to the upper surface 30a side than the bottom surface 32b, so that the gap between the aggregate 74 and the bottom surface 32b can be maintained. Therefore, even if the diameter of the aggregate 74 is large, the opening 32p in the bottom surface 32b is prevented from being partially filled with the aggregate 74.

FIG. 6C, FIG. 6D and FIG. 6E schematically show another example of the protrusions 44 of the cell culture container 50B. FIG. 6C and FIG. 6D are schematic top views of the recessed portion 32 of the cell culture container 50B, and FIG. 6E is a schematic cross-sectional view of the cell culture container 50B. As shown in FIG. 6C, FIG. 6D and FIG. 6E, the shape of the protrusions 44, the number of protrusions 44, the positions on the lateral surface 32l at which the protrusions 44 are located, etc., can be appropriately adjusted according to, for example, the diameter of the nerve cell aggregate 74 that can be cultured in the cell culture container 50B. For example, as in the example shown in FIG. 6C, the number of the plurality of protrusions 44 may be odd, such as three, five, etc. As in the example shown in FIG. 6D, the plurality of protrusions 44, each protruding from the lateral surface 32l of the recessed portion 32, may be connected together (continuous). In the example of FIG. 6D, the protrusions 44 have a generally cross shape as viewed from above. In the example of FIG. 6D, the protrusions 44 have such a shape that the protrusions 44 protruding from a plurality of points located with equal intervals along the circumference of a circle that is the intersection of the lateral surface 32l and a plane parallel to the bottom surface 32b are connected together at the center CP of the recessed portion 32 as viewed from above. Alternatively, as shown in the example of FIG. 6E, the plurality of protrusions 44 may extend from the lateral surface 32l toward the upper surface 51 (in this example, the upper surface 30a of the well member 30) side of the cell culture container 50B.

### Variation Example

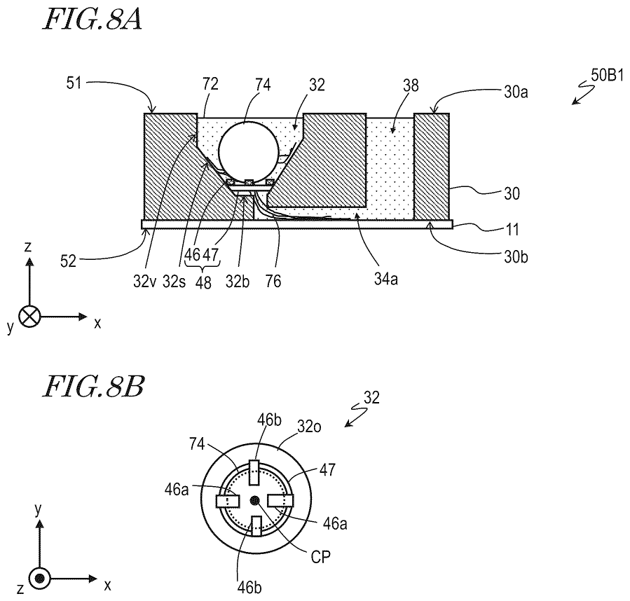

A cell culture container 50B1 according to a variation example of the present embodiment is described with reference to FIG. 8A and FIG. 8B. FIG. 8A is a schematic cross-sectional view of the cell culture container 50B1, and FIG. 8B is a schematic top view of the recessed portion 32 of the cell culture container 50B1.

The cell culture container 50B1 is different from the cell culture container 50B in that protrusions 46 are supported by a supporting portion 47 that is located in contact with the lateral surface 32l of the recessed portion 32 and that is conformable to the lateral surface 32l.

The supporting portion 47 has such a shape that the supporting portion 47 is conformable to the lateral surface 32l of the recessed portion 32 extending between the opening 32o and the bottom surface 32b as viewed from above and has a passage through which a liquid can flow. In the shown example, the supporting portion 47 has the shape of a ring. Each of the plurality of protrusions 46 supported by the supporting portion 47 in the shape of a ring extends toward the inside of the ring. The supporting portion 47 can be made of, for example, a metal such as titanium.

The protrusions 46 can have the same configuration as that of the protrusions 44 of the cell culture container 50B. Each of the protrusions 46 extends from the lateral surface 32l of the recessed portion 32 toward the inside of the recessed portion 32 in a direction parallel to the upper surface 30a (in a direction parallel to the xy plane in the drawing). The plurality of protrusions 46 include a pair of protrusions 46a that are symmetrical about the center CP of the recessed portion 32 as viewed from above. In the shown example, the plurality of protrusions 46 further include a pair of protrusions 46b that are symmetrical about the center CP of the recessed portion 32 as viewed from above.

The cell culture container 50B1 also achieves the same effects as those achieved by the cell culture container 50B. In the cell culture container 50B1, the protrusions 46 can retain the aggregate 74 at a position closer to the upper surface 30a side than the bottom surface 32b, so that the gap between the aggregate 74 and the bottom surface 32b can be maintained. Therefore, even if the diameter of the aggregate 74 is large, the opening 32p in the bottom surface 32b is prevented from being partially filled with the aggregate 74.

A member 48, which includes one or more protrusions 46 and a supporting portion 47 supporting the one or more protrusions 46, can be formed as a separate member from the well member 30. Therefore, when the shape of the protrusions 46, the number of protrusions 46, the positions on the lateral surface 32l at which the protrusions 46 are located, etc., are changed according to, for example, the diameter of the nerve cell aggregate 74 that can be cultured in the cell culture container 50B1, only the shape of the member 48 may be changed. As compared with the cell culture container 50B in which the protrusions 44 are formed integrally with the lateral surface 32l of the recessed portion 32, the manufacturing cost of the cell culture container 50B1 can be reduced.

### Embodiment 3

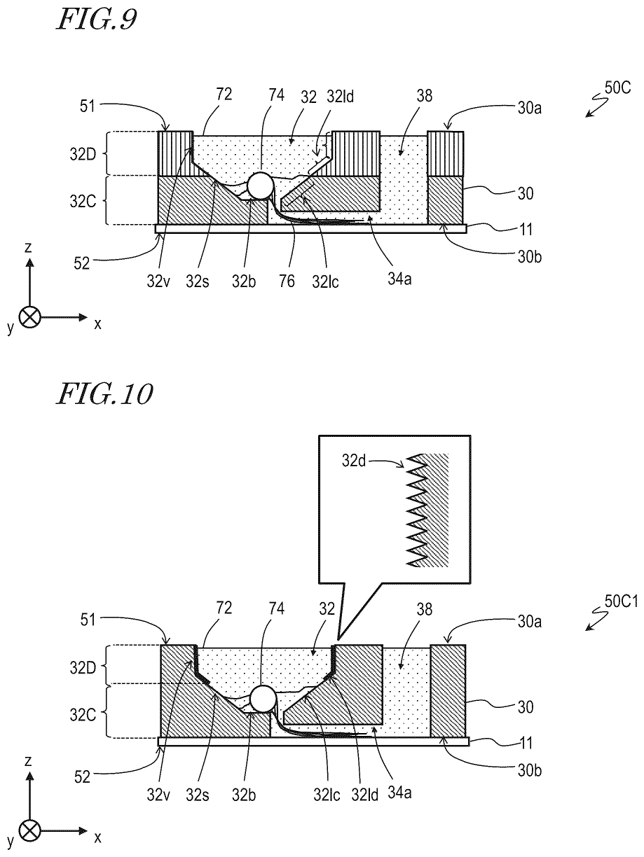

A cell culture container 50C of the present embodiment is described with reference to FIG. 9. FIG. 9 is a schematic cross-sectional view of a cell culture container 50C. Hereinafter, the differences from the previously-described embodiments are mainly described.

The cell culture container 50C is different from the cell culture container 50A in that the lateral surface 32l of the recessed portion 32 includes a first portion 32lc and a second portion 32ld located on the upper surface 30a side of the first portion 32lc, and the second portion 32ld has lower cell adhesiveness than the first portion 32lc.

Part of the cell culture container 50C which includes the first portion 32lc of the lateral surface 32l of the recessed portion 32 is referred to as the first part 32C, and another part of the cell culture container 50C which includes the second portion 32ld of the lateral surface 32l of the recessed portion 32 is referred to as the second part 32D. In this example, the well member 30 includes the first part 32C and the second part 32D. The first part 32C and the second part 32D may be made of different materials, and the second part 32D may be made of a material that has lower cell adhesiveness than the first part 32C. Alternatively, the recessed portion 32 may be made of a single material, the first portion 32lc of the lateral surface 32l may have a cell adhesion layer, which has cell adhesiveness, at its surface, and the second portion 32ld of the lateral surface 32l may not have the above-described cell adhesion layer. The cell adhesion layer can be formed by a surface treatment with a coating solution containing preference ingredients for which the cells exhibit a preference, such as extracellular matrix, and the cell adhesion layer also contains preference ingredients. Examples of the preference ingredients contained in the coating solution and the cell adhesion layer include proteins such as collagen, laminin, fibronectin, vitronectin, gelatin, etc., synthetic amino acids such as poly-D-lysine, poly-L-lysine, etc., and artificially synthesized peptides. Any one of these examples may be solely contained, or any two or more may be contained in combination.

The first part 32C and the second part 32D of the cell culture container 50C may be coincident with the first portion 32A of the recessed portion 32 which includes the slope portion 32s at the lateral surface and the second portion 32B of the recessed portion 32 which includes the vertical portion 32v at the lateral surface as illustrated with reference to FIG. 1, or may not be coincident with these portions as in the shown example.

The cell culture container 50C also enables efficient culture of nerve cells as well as the cell culture container 50A of Embodiment 1.

Further, in the cell culture container 50C, the direction in which the axons 76 extend can be more effectively controlled than in the cell culture container 50A. Since the lateral surface 32l of the recessed portion 32 includes the first portion 32lc and the second portion 32ld as previously described, the axons 76 are prevented from extending to the second portion 32ld that has relatively low cell adhesiveness. Since the axons 76 are prevented from extending to the upper surface 30a (i.e., away from the channel 34), more axons 76 can extend into the channel 34, so that a thicker bundle of the axons 76 can be formed.

### Variation Example

A cell culture container 50C1 according to a variation example of the present embodiment is described with reference to FIG. 10. FIG. 10 is a schematic cross-sectional view of the cell culture container 50C1.

In the cell culture container 50C1, the second portion 32ld of the lateral surface 32l has a minute uneven structure 32d at its surface, while the first portion 32lc of the lateral surface 32l does not have a minute uneven structure. In FIG. 10, the second portion 32ld of the lateral surface 32l is represented by a thick line, which shows that it has the minute uneven structure 32d at its surface, and a part of the second portion 32ld of the lateral surface 32l is enlargedly shown in the balloon of FIG. 10. The minute uneven structure is, for example, a moth-eye structure.

In the cell culture container 50C1, the second portion 32ld of the lateral surface 32l has the minute uneven structure 32d at its surface and hence has excellent water repellency as compared with the first portion 32lc that does not have a minute uneven structure. When a coating solution for formation of a cell adhesion layer is applied to the entirety of the lateral surface 32l of the recessed portion 32, application of the coating solution to the second portion 32ld is prevented, so that the second portion 32ld can have low cell adhesiveness as compared with the first portion 32lc. The cell culture container 50C1 also achieves the same effects as those achieved by the cell culture container 50C.

### Embodiment 4

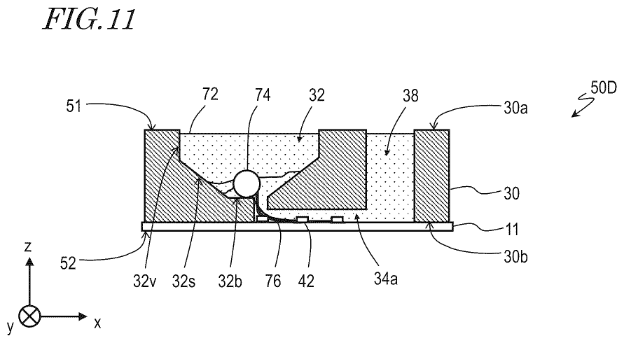

A cell culture container 50D according to the present embodiment is described with reference to FIG. 11. FIG. 11 is a schematic cross-sectional view of the cell culture container 50D. Hereinafter, the differences from the previously-described embodiments are mainly described.

The cell culture container 50D is different from the cell culture container 50A in that the cell culture container 50D further includes one or more electrodes 42 located in the first portion 34a of the channel 34. In this example, the electrodes 42 are supported by the upper surface 11a of the base 11.

The cell culture container 50D also enables efficient culture of nerve cells as well as the cell culture container 50A of Embodiment 1.

Further, in the cell culture container 50D, the direction in which the axons 76 extend can be more effectively controlled. During the culture process, by supplying a voltage to the electrodes 42, it is possible to attract the axons 76 into the first portion 34a of the channel 34. When the axons 76 are negatively charged, the axons 76 are attracted to the electrodes 42 by supplying a positive voltage to the electrodes 42.

The cell culture container 50D may include a plurality of electrodes 42. The plurality of electrodes 42 may be respectively connected with a plurality of wires that are electrically independent of one another. In such a case, it is possible to supply different voltages to the plurality of electrodes 42 and, therefore, during the culture process, the voltages can be supplied to different electrodes 42 according to the state of the axons 76. The axons 76 can be more effectively attracted into the first portion 34a of the channel 34. When the liquid culture medium 72 contains an ionic attractant, the attractant aggregates at the electrodes 42 to which the voltages are supplied, so that the effect of attracting the axons 76 can increase. The electrodes 42 may also be used to detect electrical signals (action potentials) emitted by cells during the culture process and extract the electrical signals to the outside. By controlling the direction in which the axons 76 extend, a thicker bundle of the axons 76 can be formed, so that detection of the action potentials of cells can be accurately performed.

Any two or more of previously-described Embodiments 1, 2, 3 and 4 may be employed in combination. For example, by employing Embodiment 3 and Embodiment 4 in combination, the direction in which the axons 76 extend can be more effectively controlled.

A cell culture container according to an embodiment of the present invention may include a plurality of sets of a recessed portion 32 and a channel 34 that have previously been described. For example, a cell culture container according to an embodiment of the present invention may include a base 11 and a single well member 30 supported by the base 11, and a plurality of sets of a recessed portion 32 and a channel 34 may be arrayed in the single well member 30. Alternatively, a plurality of well members 30, each of which includes a single set of a recessed portion 32 and a channel 34, may be provided and connected with one another. A cell culture container according to an embodiment of the present invention may consist only of a single well member including a plurality of sets of a recessed portion 32 and a channel 34.

A cell culture container according to an embodiment of the present invention can be used for, for example, culture of nerve cells. The cell culture container according to an embodiment of the present invention can improve the culture efficiency of nerve cells.

This application is based on Japanese Patent Application No. 2024-116796 filed on Jul. 22, 2024, the entire contents of which are hereby incorporated by reference.

What is claimed is:

1. A cell culture container comprising:
an upper surface and a lower surface located opposite to the upper surface;
a recessed portion formed in the upper surface, the recessed portion having a bottom surface that is generally parallel to the upper surface or the lower surface and a lateral surface that includes a slope portion inclined with respect to the bottom surface; and
a channel that is in communication with the recessed portion,
wherein the channel includes
a first portion extending in a direction parallel to the bottom surface and located on the lower surface side of the bottom surface, and
a second portion connecting the first portion with an opening formed in the bottom surface.

2. The cell culture container of claim 1, wherein the second portion is extending in a direction intersecting the bottom surface.

3. The cell culture container of claim 1, wherein the second portion is located so as to overlap the bottom surface as viewed from above.

4. The cell culture container of claim 1, wherein the lateral surface has one or more protrusions protruding toward an inside of the recessed portion.

5. The cell culture container of claim 4, wherein the protrusion extends from the lateral surface in a direction parallel to the bottom surface or toward the upper surface side.

6. The cell culture container of claim 4, wherein
the one or more protrusions are a plurality of protrusions, and
the plurality of protrusions are located with equal intervals at an intersection of the lateral surface and a predetermined plane that is parallel to the bottom surface.

7. The cell culture container of claim 6, wherein the plurality of protrusions include a pair of protrusions that are symmetrical about a center of the recessed portion as viewed from above.

8. The cell culture container of claim 4, wherein the protrusion is integrally formed with the lateral surface.

9. The cell culture container of claim 4, further comprising a supporting portion that is located in contact with the lateral surface of the recessed portion and that is conformable to the lateral surface,
wherein the protrusion is supported by the supporting portion.

10. The cell culture container of claim 1, wherein
the lateral surface includes a first portion and a second portion located on the upper surface side of the first portion of the lateral surface, and
the second portion of the lateral surface has lower cell adhesiveness than the first portion of the lateral surface.

11. The cell culture container of claim 10, wherein
the second portion of the lateral surface has a minute uneven structure at its surface, and
the first portion of the lateral surface does not have a minute uneven structure at its surface.

12. The cell culture container of claim 10, wherein
the first portion of the lateral surface has a cell adhesion layer, which has cell adhesiveness, at its surface, and
the second portion of the lateral surface does not have the cell adhesion layer at its surface.

13. The cell culture container of claim 10, wherein a part including the second portion of the lateral surface is made of a material that has lower cell adhesiveness than a part including the first portion of the lateral surface.

14. The cell culture container of claim 1, further comprising one or more electrodes located in the first portion of the channel.

15. The cell culture container of claim 1, further comprising
a base having an upper surface, and
a well member supported by the upper surface of the base, the well member including the recessed portion and the channel,
wherein the upper surface of the base forms a bottom of the first portion of the channel.
